(19) United States
(12) Patent Application Publication
(10) Pub. No.: US 2017/0143812 A1
(43) Pub. Date: May 25, 2017

(54) EXOSOME MEDIATED INNATE AND ADAPTIVE IMMUNE STIMULATION FOR TREATMENT OF CANCER

(71) Applicant: Therapeutic Solutions International, Inc., Oceanside, CA (US)

(72) Inventors: Thomas E. Ichim, San Diego, CA (US); Michael G. Agadjanyan, Huntington Beach, CA (US); Anahit Ghochikyan, Huntington Beach, CA (US); Santosh Kesari, Pacific Palisades, CA (US)

(21) Appl. No.: 15/349,337

(22) Filed: Nov. 11, 2016

Related U.S. Application Data
(60) Provisional application No. 62/258,007, filed on Nov. 20, 2015.

(51) Int. Cl. A61K 39/00

(52) U.S. Cl. CPC A61K 39/0011; A61K 2039/55555

(57) ABSTRACT

Disclosed are means of stimulating innate and/or adaptive immunity to cancer by administration of exosomes. Stimulation of innate immunity involves modifying exosomes by chemical addition of innate immune stimulators, whereas stimulation of adaptive immunity involves pulsing dendritic cells generating exosomes with antigens, in some cases, pulsing with Brother of the Regulator of Imprinted Sites (BORIS) proteins, peptides, or altered peptide ligands thereof.

### CROSS-REFERENCE TO RELATED APPLICATIONS

The present application claims the benefit of priority to U.S. Provisional Patent Application No. 62/258,007, filed Nov. 20, 2015, the disclosure of which is incorporated by reference herein in its entirety.

### FIELD

The present disclosure relates in general to methods of stimulating an immune response. Specifically, the present disclosure relates to methods of stimulating an immune response to cancer by administration of exosomes to a subject in need thereof. Also provided are methods of generating exosomes for administration to a subject in need, and compositions and formulations comprising the exosomes.

### BACKGROUND

Membrane microvesicles (MMV) are fragments of phospholipid bilayer plasma membrane ranging from 30 nm to 1,000 nm shed from almost all cell types. MMV, therefore are a subtype of membrane-vesicles, and play a role in intercellular communication and can deliver mRNA, siRNA, and proteins between cells. They have been implicated in the process of cancer tumor immune suppression, metastasis, tumor-stroma interactions and angiogenesis, and also play a role in tissue regeneration. They originate directly from the plasma membrane of the cell and reflect the antigenic content of the cells from which they originate.

In contrast to MMV, exosomes are vesicles of 30-100 nm in diameter, and are actively secreted by a wide range of cell types under both normal and pathological conditions. Exosomes can be regarded as a sub-class of MMV. First discovered in maturing mammalian reticulocytes, they were shown to be a mechanism for selective removal of many plasma membrane proteins and to discard transferrin-receptors from the cell surface of maturing reticulocytes. Although the exosomal protein composition varies with the cell of origin, most exosomes contain the soluble protein Hsc 70 and many others. Thirty-one proteins are found to be in common between colorectal cancer, mast cells, and urine-derived exosomes. Certain cells of the immune system, such as dendritic cells and B cells, secrete exosomes that many scientists believe play a functional role in mediating adaptive immune responses to pathogens and tumors.

Exosomes are typically formed through inward budding of endosomal membranes giving rise to intracellular multivesicular bodies (MVB) that later fuse with the plasma membrane, releasing the exosomes to the exterior. In other words, an exosome is created intracellularly when a segment of the cell membrane spontaneously invaginates and is endocytosed. The internalized segment may be broken into smaller vesicles that are subsequently expelled from the cell. The latter stage occurs when the late endosome, containing many small vesicles, fuses with the cell membrane, triggering the release of the vesicles from the cell. The vesicles (once released may be called exosomes) consist of a lipid raft embedded with ligands common to the original cell membrane. However, a more direct release of exosomes has been described, Jurkat T-cells, are said to shed exosomes directly by outward budding of the plasma membrane. Exosomes secreted by cells under normal and pathological conditions contain proteins and functional RNA molecules including mRNA and siRNA, which can be shuttled from one cell to another, affecting the recipient cell's protein production. This RNA is called “exosomal shuttle RNA”. Exosomes can also be released into urine by the kidneys and their detection might serve as a diagnostic tool. Urinary exosomes may be useful as treatment response markers in prostate cancer.

BORIS (Brother of the Regulator of Imprinted Sites) is a tumor-associated antigen, which is activated in a wide range of human cancers. In fact, aberrant synthesis of the BORIS gene product has been found in over 300 primary tumors and cancer cell lines representing all major types of human cancers with recurrent 20q13 chromosomal gains. BORIS activation has also been found in all of the standard NCI-60 cancer cell lines, which are maintained by the National Cancer Institute (NCI), and which are thought to be a reasonably complete representative set of human cancers. BORIS also is a CTCF paralog, which contains all eleven zinc fingers of CTCF, and has been shown to promote cell growth leading to transformation (see Loukinov et al., Proc. Natl. Acad. Sci. (USA) 99, 6806-6811 (2002), and International Patent Application Publication WO 03/072799 (PCT/US03/05186)). BORIS has, therefore, also been referred to as “CTCF-like” or “CTCFL” protein. One mechanism of action by which BORIS is thought to cause cancer through interference with the maintenance of an appropriate methylation pattern in the genome mediated by CCCTC binding factor (CTCF) (see Klenova et al., Seminars in Cancer Biology 12, 399-414 (2002)). The BORIS gene is believed to map to the cancer-associated amplification region of chromosome 20q13.

Although exosomes have been useful for stimulation of immunity, current means of targeting factors found in all the majority of cancer cells do not exist at present. Furthermore, conventional means of exosome utilization in immunotherapy are not practical for widespread use. The current disclosure seeks to overcome these limitations.

### SUMMARY

It is therefore an aspect of this disclosure to provide means of stimulating innate or adaptive immunity to cancer by administration of exosomes.

Some embodiments disclosed herein relate to a method of stimulating adaptive immunity. In some embodiments, the method includes pulsing dendritic cells generating exosomes with antigens. In some embodiments, pulsing dendritic cells includes pulsing with Brother of the Regulator of Imprinted Sites (BORIS) proteins, peptides, or altered peptide ligands thereof.

Some embodiments disclosed herein relate to a method of stimulating an immune response to cancer. In some embodiments, the method includes obtaining an antigen presenting cell having a potential to release exosomes and having antigen processing activity, pulsing the antigen presenting cell with a tumor antigen, collecting exosomes generated from the antigen presenting cell, and administering the exosomes to a patient in need thereof. In some embodiments, the antigen presenting cell is selected from the group including or consisting of, for example, dendritic cells, B cells, monocytes, macrophages, genetically modified cells, and mesenchymal stem cells. In some embodiments, the genetically modified cells possess antigen presenting activity, phagocytic activity, or exosome release ability.

In some embodiments, pulsing the antigen presenting cell with tumor antigen includes administering to the antigen presenting cell the tumor antigen in the form of a recombinant protein, a hybrid recombinant protein, a peptide, an altered peptide ligand, an mRNA transcript, or a plasmid encoding said antigen or an immunogenic component thereof. In some embodiments, the tumor antigen is selected from the group consisting of EGFRvIII, EGFR, HER-2, HER-3, HER-4, MET, cKit, PDGFR, Wnt, beta-catenin, K-ras, H-ras, N-ras, Raf, N-myc, c-myc, IGFR, IGFR, PI3K, Akt, tumor suppressor proteins, cancer-related host receptors, and microvesicle-associated molecules. In some embodiments, the tumor antigen is Brother of the Regulator of Imprinted Sites (BORIS). In some embodiments, the BORIS is genetically modified to lack one or more zinc finger domains. In some embodiments, the tumor suppressor proteins include BRCA1, BRCA2, or PTEN. In some embodiments, the microvesicle-associated molecules include molecules associated with angiogenesis. In some embodiments, the molecules associated with angiogenesis include VEGFR-1, VEGFR-2, Tie-2, TEM-1, or CD276.

In some embodiments, the method of stimulating an immune response to cancer includes the step of concentrating the exosomes. In some embodiments, the exosomes are concentrated by an affinity means, centrifugation, chromatography, clarification, ultrafiltration, or nanofiltration.

### DETAILED DESCRIPTION

Unless defined otherwise, all technical and scientific terms used herein have the same meaning as is commonly understood by one of ordinary skill in the art. All patents, applications, published applications, and other publications referenced herein are expressly incorporated by reference in their entireties unless stated otherwise. In the event that there is a plurality of definitions for a term herein, those in this section prevail unless stated otherwise.

The disclosure teaches means of utilizing exosomes for cancer immunotherapy. In one embodiment, the disclosure provides generation of therapeutic immune stimulating compositions through the steps of: a) obtaining immature dendritic cells; b) pulsing dendritic cells with a transcription factor associated with cancer cells; c) collecting exosomes generated by said pulsed dendritic cells; and d) administering said exosomes into a patient in need of therapy. In one embodiment said dendritic cells (DCs) are pulsed with antigen in the form of protein, peptide, altered peptide, or DNA or RNA transfection. In one embodiment of the disclosure antigen with which DCs are pulsed with is BORIS. Means of generating BORIS and BORIS with zinc finger deletions are known in the art and described in U.S. Pat. Nos. 7,579,452, and 8,114,405.

Other cancer associated proteins may be utilized for pulsing of DCs with non-limiting examples of oncogenic proteins include EGFRvIII, EGFR, HER-2, HER-3, HER-4, MET, cKit, PDGFR, Wnt, beta-catenin, K-ras, H-ras, N-ras, Raf, N-myc, c-myc, IGFR, IGFR, PI3K, and Akt; tumor suppressor proteins such as BRCA1, BRCA2 and PTEN; cancer-related host receptors and microvesicle-associated molecules, e.g. those involved in angiogenesis such as VEGFR-2, VEGFR-1, Tie-2, TEM-1 and CD276. It is contemplated that all oncogenic proteins, tumor suppressor proteins, host-cell related receptors and microvesicle-associated molecules may be used, alone or in combination, in the methods, compositions and kits of the present disclosure. It is further contemplated that any oncogenic protein, and any combination of oncogenic proteins, which is determined to be mechanistically, diagnostically, prognostically or therapeutically important for cancer, may be used in the methods, compositions and kits of the present disclosure.

Generation of dendritic cells is known in the art and may be performed according to methods described and incorporated by reference, specifically methodologies have been described for DCs generation, in which the DCs have been used in clinical trials of the following cancers: melanoma [1-52], soft tissue sarcoma [53], thyroid [54-56], glioma [57-78], multiple myeloma, [79-87], lymphoma [88-90], leukemia [91-98], as well as liver [99-104], lung [105-118], ovarian [119-122], and pancreatic cancer [123-125].

DCs were originally identified by Ralph Steinman as bone marrow derived professional antigen presenting cells, being the only cell of the immune system capable of activating naïve T cells [126]. Subsequent studies have shown that DCs act as a critical bridge between the innate immune system, which is constantly patrolling for various “danger” signals such as toll like receptor (TLR) agonists that are associated with tissue injury or pathogenic threat. In contrast to other antigen presenting cells such as the macrophage or the B cell, DCs exhibit magnitudes of higher ability to stimulate T cell responses both in antigen specific systems, as well as in polyclonal experiments such as in mixed lymphocyte reaction [127]. It is known that in peripheral tissues (outside of lymph nodes), DCs capture antigens through several complementary mechanisms including phagocytosis and receptor mediated endocytosis. Immature DCs are known to possess high degree of phagocytic activity and low levels of antigen presenting activity. Normally, DCs in peripheral tissues are immature. These immature DCs have the ability to efficiently capture antigens; they can accumulate MHC class II molecules in the late endosome-lysosomal compartment; they can express low levels of co-stimulatory molecules; they can express a unique set of chemokine receptors (such as CCR7) that allow their migration to lymphoid tissues; and they have a limited capacity for secreting cytokines [128].

Once DCs are activated, by a stimulatory signal such as a toll like receptor agonist, phagocytic activity decreases and the DCs then migrate into the draining lymph nodes through the afferent lymphatics. During the trafficking process, DCs degrade ingested proteins into peptides that bind to both WIC class I molecules and MHC class II molecules. This allows the DCs to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [129]. DCs promptly respond to environmental signals and differentiate into mature DCs that can efficiently launch immune responses. As stated above, maturation is associated with the downregulation of antigen-capture activity, the increased expression of surface MHC class II molecules and costimulatory molecules, and the ability to secrete cytokines as well as the acquisition of CCR7, which allows migration of the DCs into the draining lymph node. The ligation of the costimulatory receptor CD40 (also known as TNFRSF5) is an essential signal for the differentiation of immature DCs into fully mature DCs that are able to launch adaptive T cell-mediated immunity [130]. However, maturation of DCs alone does not result in a unique DC phenotype. Instead, the different signals that are provided by different microbes or viruses either directly or through the surrounding immune cells induce DCs to acquire distinct phenotypes that eventually contribute to different immune responses. Indeed, DC maturation varies according to different microbes because microbes express different pathogen associated molecular patterns (PAMPs) that trigger distinct DC molecular sensors, which are called pattern recognition receptors (PPRs). Strikingly, although most microbes activate DCs, a few can block DC maturation through various pathways [131]. Tissue-localized DCs can also be polarized into distinct phenotypes by the products released from surrounding immune cells that respond to injury. For example, γd-T cells and NK cells release interferon-γ (IFNγ), mast cells release pre-formed IL-4 and TNF, pDCs secrete IFNa, stromal cells secrete IL-15 and thymic stromal lymphopoietin (TSLP). These cytokines induce the differentiation of progenitor cells or of precursor cells such as monocytes into distinct inflammatory DCs that yield unique types of T cells. On interaction of CD4 and CD8 T cells with DC, these cells can subsequently differentiate into antigen-specific effector T cells with different functions. CD4 T cells can become T helper 1 (TH1) cells, TH2 cells, TH17 cells or T follicular helper (T) cells that help B cells to differentiate into antibody-secreting cells, as well as Treg cells. Naive CD8 T cells can give rise to effector cytotoxic T lymphocytes (CTLs).

An interesting activity of DCs is that in addition to stimulating immune responses through the activation of naïve T cells, DCs are also able to act as inhibitory cells. This is either directly, through inhibition of T cell activation and/or induction of T cell anergy [132], as well as indirectly through stimulation of T regulatory (Treg) cells [133, 134]. It is interesting that not only Treg cells, but also anergic T cells are capable of inhibiting DC activation [135-137], thus possibly stimulating a self-maintaining immune regulatory feedback loop. In fact, such a scenario has been previously reported where Treg stimulate immature DCs and the immature DCs in turn stimulate production of new Treg cells [138, 139].

DCs are cultured in conditions to allow for processing of exogenous antigen, such as BORIS, and subsequently exosomes are collected. The disclosure describes new processes for the preparation (i.e. isolation and/or purification) of membrane vesicles under conditions compatible with an industrial use and pharmacological applications. In particular, the processes according to the disclosure may be applied both for individualized autologous exosome preparations and for exosome preparations obtained from established cell lines, for experimental or biological use or prophylactic or therapeutic vaccination purposes, for example. This disclosure is more specifically based on the use of chromatography separation methods for preparing membrane vesicles, particularly to separate the membrane vesicles from potential biological contaminants. More specifically, a first object resides in a method of preparing membrane vesicles from a biological sample, characterized in that it comprises or includes at least an anion exchange chromatography treatment step of the sample.

Indeed, the applicant has now demonstrated that membrane vesicles, particularly exosomes, could be purified by anion exchange chromatography. In this way, unexpectedly, it is demonstrated in this application that exosomes are resolved in a homogeneous peak after anion exchange chromatography. This result is completely unexpected given that exosomes are complex supramolecular objects composed, among other things, of a membrane, surrounding an internal volume including soluble proteins. In addition, exosomes contain membrane proteins.

Therefore, a preferred aspect relates to a method of preparing, particularly of purifying, vesicle membranes, from a biological sample, including at least one anion exchange chromatography step.

To apply the method, a strong or weak, preferably strong, anion exchange may be performed. In addition, in a specific embodiment, the chromatography is performed under pressure. In some embodiments the method may be performed with high performance liquid chromatography (HPLC).

Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE.POROS®, SEPHAROSE®, SEPHADEX®, TRISACRYL®, TSK-GEL SW or PW®, SUPERDEX®TOYOPEARL HW and SEPHACRYL®, for example, which are suitable for the application of this method.

Therefore, in a specific embodiment, a method of preparing membrane vesicles from a biological sample is disclosed. The method may include, for example, at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the disclosure, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the methods of the present disclosure, it is preferable to use high porosity gels, particularly between 10 nm and 5 μm, more preferably between approximately 20 nm and approximately 2 μm, even more preferably between about 100 nm and about 1 μm.

For the anion exchange chromatography, the support used must be functionalized using a group capable of interacting with an anionic molecule. Generally, this group is composed of an amine which may be ternary or quaternary, which defines a weak or strong anion exchanger, respectively.

Within the scope of this disclosure, it is particularly advantageous to use a strong anion exchanger. In this way, according to the disclosure, a chromatography support as described above, functionalized with quaternary amines, is used. Therefore, according to a more specific embodiment of the disclosure, the anion exchange chromatography is performed on a support functionalized with a quaternary amine. Even more preferably, this support should be selected from poly(styrene-divinylbenzene), acrylamide, agarose, dextran and silica, alone or in mixtures, and functionalized with a quaternary amine.

Examples of supports functionalized with a quaternary amine include the gels SOURCEQ. MONO Q, Q SEPHAROSE®, POROS® HQ and POROS® QE, FRACTOGEL®TMAE type gels and TOYOPEARL SUPER®Q gels.

A particularly preferred support to perform the anion exchange chromatography comprises poly(styrene-divinylbenzene). An example of this type of gel which may be used within the scope of this disclosure is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size.

The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectrophotometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 μl up to 10 ml or greater. In this way, the supports available have a capacity which may reach 25 mg of proteins/ml, for example. For this reason, a 100 μl column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example.

In addition, to perform methods of this disclosure, it is also possible to combine the anion exchange chromatography step with a gel permeation chromatography step. In this way, according to a specific embodiment, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX®200HR (Pharmacia), TSK G6000 (TosoHaas) or SEPHACRYL® S (Pharmacia) is preferably used.

The process according to some embodiments of the disclosure may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the disclosure, the biological sample is a culture supernatant of membrane vesicle-producing cells.

In addition, according to a preferred embodiment of the disclosure, the biological sample is treated, prior to the chromatography step, to be enriched with membrane vesicles (enrichment stage). In this way, in a specific embodiment, this disclosure relates to a method of preparing membrane vesicles from a biological sample, characterized in that it includes at least: a) an enrichment step, to prepare a sample enriched with membrane vesicles, and b) a step during which the sample is treated by anion exchange chromatography and/or gel permeation chromatography.

According to a preferred embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography.

Therefore, a preferred method of preparing membrane vesicles according to this disclosure more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In other embodiments, the enrichment step comprises an affinity chromatography step, optionally preceded by a step of elimination of cells and/or cell debris (clarification). A preferred enrichment step according to certain embodiments of this disclosure includes (i) the elimination of cells and/or cell debris (clarification), (ii) a concentration and (iii) an affinity chromatography.

The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 μm, e.g. between 0.2 and 10 μm, are preferentially used. It is particularly possible to use a succession of filters with a porosity of 10 μm, 1 μm, 0.5 μm followed by 0.22 μm.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process.

The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibers (Amicon, Millipore, Sartorius, Pall, GF, Sepracor). Within the scope of the disclosure, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, dehydrogenases, clotting factors, interferons, lipoproteins, or also co-factors, etc. More preferably, the support used for this chromatography step is a support as used for the ion exchange chromatography, functionalized with a dye. As specific example, the dye may be selected from Blue SEPHAROSE® (Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in methods of the instant disclosure.

In some specific embodiments of the disclosure, the biological sample is obtained by subjecting a membrane vesicle-producing cell culture supernatant to at least one filtration stage.

In other specific embodiments of the disclosure, the biological sample is obtained by subjecting a membrane vesicle-producing cell culture supernatant to at least one centrifugation step.

In some preferred embodiments, the biological sample is obtained by subjecting a membrane vesicle-producing cell culture supernatant to at least one ultrafiltration step.

In other preferred embodiments, the biological sample is obtained by subjecting a membrane vesicle-producing cell culture supernatant to at least one affinity chromatography step.

A more specific preferred membrane vesicle preparation process within the scope of this disclosure comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample.

In a preferred embodiment, step b) above comprises a filtration of the culture supernatant, followed by an ultrafiltration, preferably tangential.

In another preferred embodiment, step b) above comprises a clarification of the culture supernatant, followed by an affinity chromatography on dye, preferably on Blue SEPHAROSE®.

In addition, after step c), the material harvested may, if applicable, be subjected to one or more additional treatment and/or filtration stages d), particularly for sterilization purposes. For this filtration treatment stage, filters with a diameter less than or equal to 0.3 μm are preferentially used, or even more preferentially, less than or equal to 0.25 μm. Such filters have a diameter of 0.22 μm, for example.

After step d), the material obtained is, for example, distributed into suitable devices such as bottles, tubes, bags, syringes, etc., in a suitable storage medium. The purified vesicles obtained in this way may be stored cold, frozen or used extemporaneously.

Therefore, a specific preparation process within the scope of the disclosure includes the following steps: c) an anion exchange chromatography and/or gel permeation chromatography treatment of the biological sample, and d) a filtration step, particularly sterilizing filtration, of the material harvested after stage c).

In a first variant, the process according to the disclosure comprises: c) an anion exchange chromatography treatment of the biological sample, and d) a filtration step, particularly sterilizing filtration, on the material harvested after step c).

In another variant, the process according to the disclosure comprises: c) a gel permeation chromatography treatment of the biological sample, and d) a filtration step, particularly sterilizing filtration, on the material harvested after step c).

According to a third variant, the process according to the disclosure comprises: c) an anionic exchange treatment of the biological sample followed or preceded by gel permeation chromatography, and d) a filtration step, particularly sterilizing filtration, on the material harvested after step c).

In some embodiments addition of proteins to stimulate antigen presentation and immune activation by DC derived exosomes are disclosed. Methods are known for isolation and identification a MMV fraction by employing reactants that comprise a lipophilic membrane anchoring domain and a hydrophilic target domain, such as GPI-linked proteins, to modify the MMV outer surface, or rather the vesicles' membranes, thereby providing a painting, tagging or labeling of all MMV, including exosomes, independent of their protein constitution at the outer surface of the vesicle membrane. The method is characterized therein that the hydrophilic part of the reactant which is exposed to the surrounding environment of the MMV carries a certain type of tag, such as a peptide or protein tag, which can be used to isolate the MMV it is attached to. The method is based on a reaction between a molecule referred to as the reactant, which preferably is a GPI-anchored protein, and the MMV membrane. That way it is ensured that indeed all membrane vesicles will be tagged. Because the method according to the disclosure is based on a general mode of action of the insertion of glycosyl-phosphatidyl-inositol-like anchored molecules into the membranes of all MMV, there is no limitation of the method as to certain types of MMV that do express specific surface molecules. By incubating the MMV, which preferably are exosomes, with a reactant which comprises a lipophilic membrane anchoring domain and a hydrophilic target domain and/or a protein or peptide tag, all the MMV are now tagged. As a result of this or as a result of the tag being attached to this hydrophilic target domain, they can easily be isolated or purified from the reaction mix. It is also a preferred embodiment wherein the tag at the reactant, whether bound to the amphiphilic membrane anchor domain, or bound to the hydrophilic target domain, has magnetic beads attached to it. Hence while incubating the MMV in the sample with that type of reactant; the magnetic beads are attached to the vesicles membranes in a one-step reaction. In another embodiment the Ex-tag facilitates the binding or attachment of the reactant to a membrane surface, such as metal chelate adsorber membrane (for example, Sartobind's metal chelate absorbers). Metal chelate absorbers represent Immobilized Metal Affinity Chromatography (IMAC) purification devices. They can simply be used in an HPLC, FPLC or operated by hand with a syringe connected via Luer Lock. These IMAC devices can be attached to the reactant via a suitable Ex-tag, such a polyhistidine, because histidine containing proteins bind to immobilized metal ions. Especially strong interactions take place with the commonly used polyhistidine (His 6-tag) with six consecutive histidine residues. The MMV can now be incubated with a reactant which carries a polyhistidine as EX-tag and is therefore attached to the membrane and can be concentrated or isolated from cell lysates or culture supernatant, by incubating and filtrating.

In one embodiment, therefore, is disclosed a method for exogenously modifying the membrane composition of a MMV so as to allow for binding of proteins that increase immunogenicity such as HMGB1, comprising the steps of incubating an aqueous sample that comprises MMV with a reactant comprising a hydrophilic target domain or moiety covalently linked to an amphiphilic membrane anchor domain comprising of a lipophilic part and a hydrophilic part, wherein the lipophilic part of the membrane anchor domain becomes integrated into the lipid double layer of the membrane and wherein the hydrophilic part of the anchor domain, as well as the hydrophilic target domain become exposed to the surrounding aqueous sample fluid, and wherein the hydrophilic target domain or the hydrophilic part of the anchor domain carries an EX-tag. Such EX-tag may be a peptide a protein tag, which again may be attached to magnetic beads. Preferably the EX-tag is a protein or peptide tag. A preferred protein or peptide tag in the embodiments described below would be a Histidine-tag (His-tag), FLAG-tag, Strep-tag, FLAG-tag, GST-tag, a Myc-tag, a HA-tag or an OMP A-tag or other single or several amino acid(s) that can be chemically modified to allow attachment of a second peptide or protein tag or bioactive molecule, or a chemical entity or an organic or non-organic micro- or nano- or bead or related type of particle, characterized as enabling the isolation of the so-tagged MMV (or enveloped virus particle). A peptide tag herein is understood to comprise of at least one amino acid. The stretch of amino acids (sequence) of the peptide tag is characterized as enabling the binding to a biological, chemical or metal-based or metal-related reagent, designed for the purpose of binding to the tag and thereby allowing the isolation of the MMV. This amino acid stretch may comprise a histidine (His-) tag, a Flag-tag, a strep-tag, a one Strep-tag, a GST-tag, a Myc-tag, a HA-tag or an OMP A tag, or other amino acid(s) which enable attachment of a second peptide or protein tag. It is a preferred embodiment wherein the EX-tag is an epitope tag. The epitope tag allows the according antibody to find the protein, or in this case the anchored protein, i.e. the membrane-modified vesicle, enabling lab techniques for localization, purification, and further molecular characterization. Common epitopes used for this purpose are c-myc, HA, FLAG-tag, GST and 6× or 10× His.

In one embodiment, DCs are pulsed with cancer cell lines, or lysates thereof. Said cancer cell lines may be generated by the practitioner, or may be selected from a group consisting of: K-562, THP-1 J82, RT4, ScaBER, T24, TCCSUP, 5637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-157 Breast medulla, carcinoma, pleural effusion, MDA-MB-175-VII Breast, ductal carcinoma, pleural Effusion, MDA-MB-361 Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph node ME-180 Epidermoid carcinoma, cervix, metastasis to omentum, MEL-175 Melanoma, MEL-290 Melanoma, HLA-A*0201 Melanoma cells, MS751 Epidermoid carcinoma, cervix, metastasis to lymph Node, SiHa Squamous carcinoma, cervix, JEG-3 Choriocarcinoma, Caco-2 Adenocarcinoma, colon HT-29 Adenocarcinoma, colon, moderately well-differentiated grade II, SK-CO-1 Adenocarcinoma, colon, ascites, HuTu 80 Adenocarcinoma, duodenum, A-253 Epidermoid carcinoma, submaxillary gland FaDu Squamous cell carcinoma, pharynx, A-498 Carcinoma, kidney, A-704 Adenocarcinoma, kidney Adenocarcinoma, kidney, SK-HEP-1 Adenocarcinoma, liver, ascites, A-427 Carcinoma, lung, Caki-1 Clear cell carcinoma, consistent with renal primary, metastasis to skin, Caki-2 Clear cell carcinoma, consistent with renal primary, SK-NEP-1 Wilms' tumor, pleural effusion, SW 839 Adenocarcinoma, kidney, SK-HEP-1 Adenocarcinoma, liver, ascites, A-427 Carcinoma, lung Calu-1 Epidermoid carcinoma grade III, lung, metastasis to pleura, Calu-3 Adenocarcinoma, lung, pleural effusion, Calu-6 Anaplastic carcinoma, probably lung, SK-LU-1 Adenocarcinoma, lung consistent with poorly differentiated, grade III, SK-MES-1 Squamous carcinoma, lung, pleural effusion, SW 900 Squamous cell carcinoma, lung, EB1 Burkitt lymphoma, upper maxilia, EB2 Burkitt lymphoma, ovary P3HR-1 Burkitt lymphoma, ascites, HT-144 Malignant melanoma, metastasis to subcutaneous tissue Malme-3M Malignant melanoma, metastasis to lung, RPMI-7951 Malignant melanoma, metastasis to lymph node, SK-MEL-1 Malignant melanoma, metastasis to lymphatic system, SK-MEL-2 Malignant melanoma, metastasis to skin of thigh, SK-MEL-3 Malignant melanoma, metastasis to lymph node SK-MEL-5 Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1 Adenocarcinoma, pancreas, metastasis to liver, Capan-2 Adenocarcinoma, pancreas, DU 145 Carcinoma, prostate, metastasis to brain, A-204 Rhabdomyosarcoma, Saos-2 Osteogenic sarcoma, primary, SK-ES-1 Anaplastic osteosarcoma versus Swing sarcoma, SK-LNS-1 Leiomyosarcoma, vulva, primary, SW 684 Fibrosarcoma, SW 872 Liposarcoma, SW 982 Axilla synovial sarcoma, SW 1353 Chondrosarcoma, humerus, U-2 OS Osteogenic sarcoma, bone primary, Malme-3 Skin fibroblast, KATO III Gastric carcinoma, Cate-1B Embryonal carcinoma, testis, metastasis to lymph node, Tera-1 Embryonal carcinoma, Tera-2 Embryonal carcinoma, SW579 Thyroid carcinoma, AN3 CA Endometrial adenocarcinoma, metastatic, HEC-1-A Endometrial adenocarcinoma, HEC-1-B Endometrial adenocarcinoma, SK-UT-1 Uterine, mixed mesodermal tumor, consistent with leiomyosarcoma grade III, SK-UT-1B Uterine, mixed mesodermal tumor, Sk-Me128 Melanoma, SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast BT-549 Ductal carcinoma, breast, DU4475 Metastatic cutaneous nodule, breast carcinoma HBL-100 Breast, Hs 578Bst Breast, Hs 578T Ductal carcinoma, breast, MDA-MB-330 Carcinoma, breast MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738B1 Bladder, normal fetus NIH:OVCAR-3 Ovary, adenocarcinoma, Hs 67 Thymus, normal, RD-ES Ewing's sarcoma ChaGo K-1 Bronchogenic carcinoma, subcutaneous, metastasis, human, WERI-Rb-1 Retinoblastoma NCI-H446 Small cell carcinoma, lung, NCI-H209 Small cell carcinoma, lung, NCI-H146 Small cell carcinoma, lung, NCI-H441 Papillary adenocarcinoma, lung, NCI-H345 Small cell carcinoma, lung, NCI-H820 Papillary adenocarcinoma, lung, NCI-H520 Squamous cell carcinoma, lung, NCI-H661 Large cell carcinoma, lung NCI-H510A Small cell carcinoma, extra-pulmonary origin, metastatic D283 Med Medulloblastoma Daoy Medulloblastoma, D341 Med Medulloblastoma, AML-193 Acute monocyte leukemia MV4-11 Leukemia biphenotype, NCI-H82 Small cell carcinoma, lung H9 T-cell lymphoma, NCI-H460 Large cell carcinoma, lung, NCI-H596 Adenosquamous carcinoma, or lung NCI-H676B Adenocarcinoma of lung.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the claims.

### REFERENCES, EACH OF WHICH IS EXPRESSLY INCORPORATED BY REFERENCE IN ITS ENTIRETY HEREIN

1. Nestle, F. O., et al., Vaccination of melanoma patients with peptide-or tumor lysate-pulsed dendritic cells. Nat Med, 1998. 4(3): p. 328-32.
2. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
3. Wang, F., et al., Phase I trial of a MART-1 peptide vaccine with incomplete Freund's adjuvant for resected high-risk melanoma. Clin Cancer Res, 1999. 5(10): p. 2756-65.
4. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
5. Thomas, R., et al., Immature human monocyte-derived dendritic cells migrate rapidly to draining lymph nodes after intradermal injection for melanoma immunotherapy. Melanoma Res, 1999. 9(5): p. 474-81.
6. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
7. Panelli, M. C., et al., Phase 1 study in patients with metastatic melanoma of immunization with dendritic cells presenting epitopes derived from the melanoma-associated antigens MART-1 and gp100. J Immunother, 2000. 23(4): p. 487-98.
8. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
9. Lau, R., et al., Phase I trial of intravenous peptide pulsed dendritic cells in patients with metastatic melanoma. J Immunother, 2001. 24(1): p. 66-78.
10. Banchereau, J., et al., Immune and clinical responses in patients with metastatic melanoma to CD34(+) progenitor-derived dendritic cell vaccine. Cancer Res, 2001. 61(17): p. 6451-8.
11. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
12. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
13. Bedrosian, I., et al., Intranodal administration of peptide-pulsed mature dendritic cell vaccines results in superior CD8+T-cell function in melanoma patients. J Clin Oncol, 2003. 21(20): p. 3826-35.
14. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
15. Hersey, P., et al., Phase I/II study of treatment with dendritic cell vaccines in patients with disseminated melanoma. Cancer Immunol Immunother, 2004. 53(2): p. 125-34.
16. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
17. Palucka, A. K., et al., Spontaneous proliferation and type 2 cytokine secretion by CD4+T cells in patients with metastatic melanoma vaccinated with antigen pulsed dendritic cells. J Clin Immunol, 2005. 25(3): p. 288-95.
18. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.
19. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
20. Salcedo, M., et al., Vaccination of melanoma patients using dendritic cells loaded with an allogeneic tumor cell lysate. Cancer Immunol Immunother, 2006. 55(7): p. 819-29.
21. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
22. Escobar, A., et al., Dendritic cell immunizations alone or combined with low doses of interleukin-2 induce specific immune responses in melanoma patients. Clin Exp Immunol, 2005. 142(3): p. 555-68.
23. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
24. Schadendorf, D., et al., Dacarbazine (DTIC) versus vaccination with autologous peptide pulsed dendritic cells (DC) in first-line treatment of patients with metastatic melanoma: a randomized phase III trial of the DC study group of the DeCOG. Ann Oncol, 2006. 17(4): p. 563-70.
25. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
26. Nakai, N., et al., Vaccination of Japanese patients with advanced melanoma with peptide, tumor lysate or both peptide and tumor lysate pulsed mature, monocyte-derived dendritic cells. J Dermatol, 2006. 33(7): p. 462-72.
27. Palucka, A. K., et al., Dendritic cells loaded with killed allogeneic melanoma cells can induce objective clinical responses and MART-1 specific CD8+ T-cell immunity. J Immunother, 2006. 29(5): p. 545-57.
28. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
29. Guo, J., et al., Intratumoral injection of dendritic cells in combination with local hyperthermia induces systemic antitumor effect in patients with advanced melanoma. Int J Cancer, 2007. 120(11): p. 2418-25.
30. O'Rourke, M. G., et al., Dendritic cell immunotherapy for stage IV melanoma. Melanoma Res, 2007. 17(5): p. 316-22.
31. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
32. Hersey, P., et al., Phase I/II study of treatment with matured dendritic cells with or without low dose IL-2 in patients with disseminated melanoma. Cancer Immunol Immunother, 2008. 57(7): p. 1039-51.
33. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
34. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
35. Redman, B. G., et al., Phase Ib trial assessing autologous, tumor pulsed dendritic cells as a vaccine administered with or without IL-2 in patients with metastatic melanoma. J Immunother, 2008. 31(6): p. 591-8.
36. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
37. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
38. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
39. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
40. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
41. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
42. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
43. Ribas, A., et al., Multicenter phase II study of matured dendritic cells pulsed with melanoma cell line lysates in patients with advanced melanoma. J Transl Med, 2010. 8: p. 89.
44. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
45. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
46. Lesterhuis, W. J., et al., Wild-type and modified gp100 peptide pulsed dendritic cell vaccination of advanced melanoma patients can lead to long-term clinical responses independent of the peptide used. Cancer Immunol Immunother, 2011. 60(2): p. 249-60.
47. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
48. Steele, J. C., et al., Phase I/II trial of a dendritic cell vaccine transfected with DNA encoding melan A and gp100 for patients with metastatic melanoma. Gene Ther, 2011. 18(6): p. 584-93.
49. Kim, D. S., et al., Immunotherapy of malignant melanoma with tumor lysate-pulsed autologous monocyte-derived dendritic cells. Yonsei Med J, 2011. 52(6): p. 990-8.
50. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
51. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.
52. Dannull, J., et al., Melanoma immunotherapy using mature DCs expressing the constitutive proteasome. J Clin Invest, 2013. 123(7): p. 3135-45.
53. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
54. Stift, A., et al., Dendritic cell vaccination in medullary thyroid carcinoma. Clin Cancer Res, 2004. 10(9): p. 2944-53.
55. Kuwabara, K., et al., Results of a phase I clinical study using dendritic cell vaccinations for thyroid cancer. Thyroid, 2007. 17(1): p. 53-8.
56. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
57. Yu, J. S., et al., Vaccination of malignant glioma patients with peptide pulsed dendritic cells elicits systemic cytotoxicity and intracranial T-cell infiltration. Cancer Res, 2001. 61(3): p. 842-7.
58. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
59. Yu, J. S., et al., Vaccination with tumor lysate-pulsed dendritic cells elicits antigen-specific, cytotoxic T-cells in patients with malignant glioma. Cancer Res, 2004. 64(14): p. 4973-9.
60. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.
61. Yamanaka, R., et al., Clinical evaluation of dendritic cell vaccination for patients with recurrent glioma: results of a clinical phase I/II trial. Clin Cancer Res, 2005. 11(11): p. 4160-7.
62. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
63. Walker, D. G., et al., Results of a phase I dendritic cell vaccine trial for malignant astrocytoma: potential interaction with adjuvant chemotherapy. J Clin Neurosci, 2008. 15(2): p. 114-21.
64. Leplina, O. Y., et al., Use of interferon-alpha-induced dendritic cells in the therapy of patients with malignant brain gliomas. Bull Exp Biol Med, 2007. 143(4): p. 528-34.
65. De Vleeschouwer, S., et al., Postoperative adjuvant dendritic cell-based immunotherapy in patients with relapsed glioblastoma multiforme. Clin Cancer Res, 2008. 14(10): p. 3098-104.
66. Ardon, H., et al., Adjuvant dendritic cell-based tumour vaccination for children with malignant brain tumours. Pediatr Blood Cancer, 2010. 54(4): p. 519-25.
67. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
68. Okada, H., et al., Induction of CD8+ T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
69. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
70. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
71. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
72. Iwami, K., et al., Peptide pulsed dendritic cell vaccination targeting interleukin-13 receptor alpha2 chain in recurrent malignant glioma patients with HLA-A*24/A*02 allele. Cytotherapy, 2012. 14(6): p. 733-42.
73. Fong, B., et al., Monitoring of regulatory T cell frequencies and expression of CTLA-4 on T cells, before and after DC vaccination, can predict survival in GBM patients. PLoS One, 2012. 7(4): p. e32614.
74. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
75. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
76. Akiyama, Y., et al., alpha-type-1 polarized dendritic cell-based vaccination in recurrent high-grade glioma: a phase I clinical trial. BMC Cancer, 2012. 12: p. 623.
77. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
78. Shah, A. H., et al., Dendritic cell vaccine for recurrent high-grade gliomas in pediatric and adult subjects: clinical trial protocol. Neurosurgery, 2013. 73(5): p. 863-7.
79. Reichardt, V. L., et al., Idiotype vaccination using dendritic cells after autologous peripheral blood stem cell transplantation for multiple myeloma—a feasibility study. Blood, 1999. 93(7): p. 2411-9.
80. Lim, S. H. and R. Bailey-Wood, Idiotypic protein-pulsed dendritic cell vaccination in multiple myeloma. Int J Cancer, 1999. 83(2): p. 215-22.
81. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
82. Reichardt, V. L., et al., Idiotype vaccination of multiple myeloma patients using monocyte-derived dendritic cells. Haematologica, 2003. 88(10): p. 1139-49.
83. Guardino, A. E., et al., Production of myeloid dendritic cells (DC) pulsed with tumor-specific idiotype protein for vaccination of patients with multiple myeloma. Cytotherapy, 2006. 8(3): p. 277-89.
84. Lacy, M. Q., et al., Idiotype-pulsed antigen-presenting cells following autologous transplantation for multiple myeloma may be associated with prolonged survival. Am J Hematol, 2009. 84(12): p. 799-802.
85. Yi, Q., et al., Optimizing dendritic cell-based immunotherapy in multiple myeloma: intranodal injections of idiotype pulsed CD40 ligand-matured vaccines led to induction of type-1 and cytotoxic T-cell immune responses in patients. Br J Haematol, 2010. 150(5): p. 554-64.
86. Rollig, C., et al., Induction of cellular immune responses in patients with stage-I multiple myeloma after vaccination with autologous idiotype pulsed dendritic cells. J Immunother, 2011. 34(1): p. 100-6.
87. Zahradova, L., et al., Efficacy and safety of Id-protein-loaded dendritic cell vaccine in patients with multiple myeloma—phase II study results. Neoplasma, 2012. 59(4): p. 440-9.
88. Timmerman, J. M., et al., Idiotype-pulsed dendritic cell vaccination for B-cell lymphoma: clinical and immune responses in 35 patients. Blood, 2002. 99(5): p. 1517-26.
89. Maier, T., et al., Vaccination of patients with cutaneous T-cell lymphoma using intranodal injection of autologous tumor-lysate-pulsed dendritic cells. Blood, 2003. 102(7): p. 2338-44.
90. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
91. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
92. Li, L., et al., Immunotherapy for patients with acute myeloid leukemia using autologous dendritic cells generated from leukemic blasts. Int J Oncol, 2006. 28(4): p. 855-61.
93. Roddie, H., et al., Phase I/II study of vaccination with dendritic-like leukaemia cells for the immunotherapy of acute myeloid leukaemia. Br J Haematol, 2006. 133(2): p. 152-7.
94. Litzow, M. R., et al., Testing the safety of clinical-grade mature autologous myeloid DC in a phase I clinical immunotherapy trial of CML. Cytotherapy, 2006. 8(3): p. 290-8.
95. Westermann, J., et al., Vaccination with autologous non-irradiated dendritic cells in patients with bcr/abl+ chronic myeloid leukaemia. Br J Haematol, 2007. 137(4): p. 297-306.
96. Hus, I., et al., Vaccination of B-CLL patients with autologous dendritic cells can change the frequency of leukemia antigen-specific CD8+ T cells as well as CD4+CD25+FoxP3+ regulatory T cells toward an antileukemia response. Leukemia, 2008. 22(5): p. 1007-17.
97. Palma, M., et al., Development of a dendritic cell-based vaccine for chronic lymphocytic leukemia. Cancer Immunol Immunother, 2008. 57(11): p. 1705-10.
98. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
99. Iwashita, Y., et al., A phase I study of autologous dendritic cell-based immunotherapy for patients with unresectable primary liver cancer. Cancer Immunol Immunother, 2003. 52(3): p. 155-61.
100. Lee, W. C., et al., Vaccination of advanced hepatocellular carcinoma patients with tumor lysate pulsed dendritic cells: a clinical trial. J Immunother, 2005. 28(5): p. 496-504.
101. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
102. Palmer, D. H., et al., A phase II study of adoptive immunotherapy using dendritic cells pulsed with tumor lysate in patients with hepatocellular carcinoma. Hepatology, 2009. 49(1): p. 124-32.
103. El Ansary, M., et al., Immunotherapy by autologous dendritic cell vaccine in patients with advanced HCC. J Cancer Res Clin Oncol, 2013. 139(1): p. 39-48.
104. Tada, F., et al., Phase I/II study of immunotherapy using tumor antigen-pulsed dendritic cells in patients with hepatocellular carcinoma. Int J Oncol, 2012. 41(5): p. 1601-9.
105. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
106. Hirschowitz, E. A., et al., Autologous dendritic cell vaccines for non-small-cell lung cancer. J Clin Oncol, 2004. 22(14): p. 2808-15.
107. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
108. Yannelli, J. R., et al., The large scale generation of dendritic cells for the immunization of patients with non-small cell lung cancer (NSCLC). Lung Cancer, 2005. 47(3): p. 337-50.
109. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
110. Antonia, S. J., et al., Combination of p53 cancer vaccine with chemotherapy in patients with extensive stage small cell lung cancer. Clin Cancer Res, 2006. 12(3 Pt 1): p. 878-87.
111. Perrot, I., et al., Dendritic cells infiltrating human non-small cell lung cancer are blocked at immature stage. J Immunol, 2007. 178(5): p. 2763-9.
112. Hirschowitz, E. A., et al., Immunization of NSCLC patients with antigen-pulsed immature autologous dendritic cells. Lung Cancer, 2007. 57(3): p. 365-72.
113. Baratelli, F., et al., Pre-clinical characterization of GMP grade CCL21-gene modified dendritic cells for application in a phase I trial in non-small cell lung cancer. J Transl Med, 2008. 6: p. 38.
114. Hegmans, J. P., et al., Consolidative dendritic cell-based immunotherapy elicits cytotoxicity against malignant mesothelioma. Am J Respir Crit Care Med, 2010. 181(12): p. 1383-90.
115. Um, S. J., et al., Phase I study of autologous dendritic cell tumor vaccine in patients with non-small cell lung cancer. Lung Cancer, 2010. 70(2): p. 188-94.
116. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
117. Perroud, M. W., Jr., et al., Mature autologous dendritic cell vaccines in advanced non-small cell lung cancer: a phase I pilot study. J Exp Clin Cancer Res, 2011. 30: p. 65.
118. Skachkova, O. V., et al., Immunological markers of anti-tumor dendritic cells vaccine efficiency in patients with non-small cell lung cancer. Exp Oncol, 2013. 35(2): p. 109-13.
119. Hernando, J. J., et al., Vaccination with autologous tumour antigen pulsed dendritic cells in advanced gynaecological malignancies: clinical and immunological evaluation of a phase I trial. Cancer Immunol Immunother, 2002. 51(1): p. 45-52.
120. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
121. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
122. Kandalaft, L. E., et al., A Phase I vaccine trial using dendritic cells pulsed with autologous oxidized lysate for recurrent ovarian cancer. J Transl Med, 2013. 11: p. 149.
123. Lepisto, A. J., et al., A phase I/II study of a MUC1 peptide pulsed autologous dendritic cell vaccine as adjuvant therapy in patients with resected pancreatic and biliary tumors. Cancer Ther, 2008. 6(B): p. 955-964.
124. Rong, Y., et al., A phase I pilot trial of MUC1-peptide-pulsed dendritic cells in the treatment of advanced pancreatic cancer. Clin Exp Med, 2012. 12(3): p. 173-80.
125. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.
126. Steinman, R. M. and Z. A. Cohn, Identification of a novel cell type in peripheral lymphoid organs of mice. I. Morphology, quantitation, tissue distribution. J Exp Med, 1973. 137(5): p. 1142-62.
127. Banchereau, J. and R. M. Steinman, Dendritic cells and the control of immunity. Nature, 1998. 392(6673): p. 245-52.
128. Trombetta, E. S. and I. Mellman, Cell biology of antigen processing in vitro and in vivo. Annu Rev Immunol, 2005. 23: p. 975-1028.
129. Itano, A. A. and M. K. Jenkins, Antigen presentation to naive CD4 T cells in the lymph node. Nat Immunol, 2003. 4(8): p. 733-9.
130. Caux, C., et al., Activation of human dendritic cells through CD40 cross-linking. J Exp Med, 1994. 180(4): p. 1263-72.
131. Pulendran, B., K. Palucka, and J. Banchereau, Sensing pathogens and tuning immune responses. Science, 2001. 293(5528): p. 253-6.
132. Lutz, M. B., et al., Culture of bone marrow cells in GM-CSF plus high doses of lipopolysaccharide generates exclusively immature dendritic cells which induce alloantigen-specific CD4 T cell anergy in vitro. Eur J Immunol, 2000. 30(4): p. 1048-52.
133. Turnquist, H. R. and A. W. Thomson, Taming the lions: manipulating dendritic cells for use as negative cellular vaccines in organ transplantation. Curr Opin Organ Transplant, 2008. 13(4): p. 350-7.
134. Pletinckx, K., et al., Role of dendritic cell maturity/costimulation for generation, homeostasis, and suppressive activity of regulatory T cells. Front Immunol, 2011. 2: p. 39.
135. Frasca, L., et al., Human anergic CD4+ T cells can act as suppressor cells by affecting autologous dendritic cell conditioning and survival. J Immunol, 2002. 168(3): p. 1060-8.
136. Vendetti, S., et al., Anergic T cells inhibit the antigen presenting function of dendritic cells. J Immunol, 2000. 165(3): p. 1175-81.
137. Veldhoen, M., et al., Modulation of dendritic cell function by naive and regulatory CD4+ T cells. J Immunol, 2006. 176(10): p. 6202-10.
138. Guillot, C., et al., Active suppression of allogeneic proliferative responses by dendritic cells after induction of long-term allograft survival by CTLA4Ig. Blood, 2003. 101(8): p. 3325-33.
139. Roelofs-Haarhuis, K., et al., Infectious nickel tolerance: a reciprocal interplay of tolerogenic APCs and T suppressor cells that is driven by immunization. J Immunol, 2003. 171(6): p. 2863-72.

What is claimed is:

1. A method of stimulating an immune response to cancer in a patient in need thereof, the method comprising the steps of:
obtaining an antigen presenting cell having a potential to release exosomes and having antigen processing activity;
pulsing said antigen presenting cell with a tumor antigen;
collecting exosomes generated from said antigen presenting cell; and
administering said exosomes to a patient in need thereof.

2. The method of claim 1, wherein said antigen presenting cell is selected from the group consisting of dendritic cells, B cells, monocytes, macrophages, genetically modified cells and mesenchymal stem cells.

3. The method of claim 2, wherein the genetically modified cells possess antigen presenting activity, phagocytic activity, or exosome release ability.

4. The method of claim 1, further comprising concentrating said exosomes.

5. The method of claim 4, wherein said exosomes are concentrated by an affinity means, centrifugation, chromatography, clarification, ultrafiltration, or nanofiltration.

6. The method of claim 1, wherein pulsing the antigen presenting cell with tumor antigen comprises administering to said antigen presenting cell said tumor antigen in the form of a recombinant protein, a hybrid recombinant protein, a peptide, an altered peptide ligand, an mRNA transcript, or a plasmid encoding said antigen or an immunogenic component thereof.

7. The method of claim 6, wherein said tumor antigen is selected from the group consisting of EGFRvIII, EGFR, HER-2, HER-3, HER-4, MET, cKit, PDGFR, Wnt, beta-catenin, K-ras, H-ras, N-ras, Raf, N-myc, c-myc, IGFR, IGFR, PI3K, Akt, tumor suppressor proteins, cancer-related host receptors, and microvesicle-associated molecules.

8. The method of claim 6, wherein said tumor antigen is Brother of the Regulator of Imprinted Sites (BORIS).

9. The method of claim 8, wherein said BORIS is genetically modified to lack one or more zinc finger domains.

10. The method of claim 7, wherein the tumor suppressor proteins comprise BRCA1, BRCA2, or PTEN.

11. The method of claim 7, wherein the microvesicle-associated molecules comprise molecules associated with angiogenesis.

12. The method of claim 11, wherein the molecules associated with angiogenesis include VEGFR-1.

13. The method of claim 11, wherein the molecules associated with angiogenesis include VEGFR-2.

14. The method of claim 11, wherein the molecules associated with angiogenesis include Tie-2.

15. The method of claim 11, wherein the molecules associated with angiogenesis include TEM-1.

16. The method of claim 11, wherein the molecules associated with angiogenesis include CD276.
